(19) United States
(12) Patent Application Publication
(10) Pub. No.: US 2023/0135227 A1
(43) Pub. Date: May 4, 2023

(54) USE OF PHYCOBILIPROTEIN (PBP) TO INHIBIT OR TREAT SARS-COV-2 INFECTION

(71) Applicant: Back of the Yards Algae Sciences LLC, Chicago, IL (US)

(72) Inventor: Leonard Lerer, Chicago, IL (US)

(21) Appl. No.: 17/914,805

(22) PCT Filed: Mar. 25, 2021
(86) PCT No.: PCT/US2021/024040

§ 371 Date: Sep. 27, 2022

Related U.S. Application Data
(60) Provisional application No. 63/000,701, filed on Mar. 27, 2020.

(51) Int. Cl. A61K 38/16; C12N 1/12; A61K 9/00

(52) U.S. Cl. CPC A61K 38/164; C12N 1/12; A61K 9/0043

(57) ABSTRACT

The present invention relates to the use of phycobiliprotein (PBP) to inhibit or treat SARS-CoV-2 infection and COVID 19.

### CROSS REFERENCE TO RELATED APPLICATION

This application claims priority to and the benefit of U.S. Provisional Application No. 63/000,701, filed Mar. 27, 2020, which is hereby incorporated by reference in its entirety.

### FIELD OF THE INVENTION

The present invention relates to the use of phycobiliprotein (PBP) to inhibit or treat SARS-CoV-2 infection and COVID 19.

### BACKGROUND OF THE INVENTION

Coronaviruses are a large family of viruses, some causing illness in people, and others that circulate among animals, including camels, cats, and bats. Rarely animal coronaviruses can evolve and infect people and then spread between people.

Human coronaviruses are common throughout the world and commonly cause mild to moderate illness in people worldwide. However, the emergence of novel (new) coronaviruses, such as SARS and MERS, have been associated with more severe respiratory illness.

As of March 2020 WHO has declared a pandemic due the emergence of SARS-CoV-2 (Severe Acute Respiratory Syndrome Coronavirus 2) in China. The virus is now known as the severe acute respiratory syndrome coronavirus 2 (SARS-CoV-2). The disease it causes is called coronavirus disease 2019 (COVID-19). Symptoms may include fever, cough and shortness of breath. Human coronaviruses can sometimes cause lower-respiratory tract illnesses, such as pneumonia or bronchitis. Human coronaviruses can sometimes cause a severe systemic inflammatory response (cytokine release syndrome) and multi-organ failure.

What is needed in the art are safe and effective treatments for preventing or inhibiting infection by SARS-CoV-2 and/or treating or otherwise ameliorating the symptoms of COVID-19.

### SUMMARY OF THE INVENTION

The present invention relates to the use of phycobiliprotein (PBP) to inhibit or treat or provide prophylaxis for SARS-CoV-2 infection and/or COVID 19.

In some preferred embodiments, the present invention provides methods of treating COVID-19 in a subject in thereof comprising administering to a subject diagnosed with COVID-19 a therapeutically effective amount of a phycobiliprotein (PBP) composition.

In some preferred embodiments, the present invention provides methods of inhibiting SARS-CoV-2 infection in a subject in thereof comprising administering to a subject diagnosed with COVID-19 a prophylactically effective amount of a phycobiliprotein (PBP).

In some preferred embodiments, the present invention provides methods for prophylaxis of SARS-CoV-2 infection or COVID-19 in a subject in thereof comprising administering to a prophylactically effective amount of a phycobiliprotein (PBP). In some preferred embodiments, the subject has been diagnosed with COVID-19.

In some embodiments, the subjects have long COVID symptoms. In some embodiments, the subject has exhibited long COVID symptoms for at least 2 months after diagnoses of COVID 19. In some embodiments, the subject has exhibited long COVID symptoms for at least 3 months after diagnoses of COVID 19. In some embodiments, the subject has exhibited long COVID symptoms for at least 4 months after diagnoses of COVID 19. In some embodiments, the subject has exhibited long COVID symptoms for at least 5 months after diagnoses of COVID 19. In some embodiments, the subject has exhibited long COVID symptoms for at least 6 months after diagnoses of COVID 19. In some embodiments, the subject has exhibited long COVID symptoms for at least 9 months after diagnoses of COVID 19. In some embodiments, the subject has exhibited long COVID symptoms for at least 12 months after diagnoses of COVID 19. In some embodiments, the long COVID symptoms are one or more of: fatigue, loss of smell and taste, shortness of breath, cough, joint pain, chest pain, difficulty with thinking and concentration (sometimes referred to as “brain fog”), depression, headache, heart palpitations, inflammation of the heart muscle, rash, hair loss, sleep disorders, loss of lung function and loss of memory. In some preferred embodiments, administration of the PBP composition improves or alleviates one or more of the following symptoms in a long COVID patient: fatigue, loss of smell and taste, shortness of breath, cough, joint pain, chest pain, difficulty with thinking and concentration (sometimes referred to as “brain fog”), depression, headache, heart palpitations, inflammation of the heart muscle, rash, hair loss, sleep disorders, loss of lung function and loss of memory.

In some preferred embodiments, the administration of the PBP composition is sufficient to relieve or ameliorate one or more symptoms of COVID-19. The symptoms that are relieved or ameliorated include one or more of fatigue, loss of smell and taste, shortness of breath, cough, joint pain, chest pain, difficulty with thinking and concentration (sometimes referred to as “brain fog”), depression, headache, heart palpitations, inflammation of the heart muscle, rash, hair loss, sleep disorders, loss of lung function and loss of memory. In some preferred embodiments, administration of the PBP composition improves or alleviates one or more of the following symptoms in a long COVID patient: fatigue, loss of smell and taste, shortness of breath, cough, joint pain, chest pain, difficulty with thinking and concentration (sometimes referred to as “brain fog”), depression, headache, heart palpitations, inflammation of the heart muscle, rash, hair loss, sleep disorders, loss of lung function and loss of memory.

In some preferred embodiments, the subject in need thereof is selected from the group consisting of a health care workers, a doctor, a nurse, a teacher, a day-care worker, a hospital worker, elderly subjects such as those over 60, 70 or 80 years of age, an immunocompromised subject, a subject with high blood pressure, and a subject with impaired lung function. In some preferred embodiments, the subject with impaired lung function has a history of lung cancer, chronic obstructive pulmonary disease (COPD), emphysema, smoking, or vaping.

In some preferred embodiments, the present invention provides phycobiliprotein (PBP) composition for use in treating COVID-19 in a subject in need thereof.

In some preferred embodiments, the present invention provides methods phycobiliprotein (PBP) composition for use in treating or inhibiting SARS-CoV-2 infection in a subject in need thereof. In some preferred embodiments, the subject in need thereof is selected from the group consisting of a health care workers, a doctor, a nurse, a teacher, a day-care worker, a hospital worker, elderly subjects such as those over 60, 70 or 80 years of age, an immunocompromised subject, a subject with high blood pressure, and a subject with impaired lung function. In some preferred embodiments, the subject with impaired lung function has a history of lung cancer, chronic obstructive pulmonary disease (COPD), emphysema, smoking, or vaping.

In some preferred embodiments, the PBP composition utilized in any of the foregoing embodiments is a purified phycobiliprotein (PBP) composition characterized by one or more of the following characteristics:

a) the protein fraction of the composition comprises greater than about 30% of a protein having a molecular weight of about 17,695 kDa and an isoelectric point of about 6.29 as assayed by two dimensional gel electrophoresis followed by quantitation by densitometry;

b) the protein fraction of the composition comprises greater than about 5% of a protein having a molecular weight of about 19,833 kDa and an isoelectric point of about 6.14 as assayed by two dimensional gel electrophoresis followed by quantitation by densitometry;

c) the protein fraction of the composition comprises greater than about 1% of a protein having a molecular weight of about 45,578 kDa and an isoelectric point of about 6.2 as assayed by two dimensional gel electrophoresis followed by quantitation by densitometry;

d) the protein fraction of the composition comprises greater than about 1% of a protein having a molecular weight of about 35,014 kDa and an isoelectric point of about 5.9 as assayed by two dimensional gel electrophoresis followed by quantitation by densitometry;

e) the protein fraction of the composition comprises greater than about 0.30% of a protein having a molecular weight of about 24,688 kDa and an isoelectric point of about 5.3 as assayed by two dimensional gel electrophoresis followed by quantitation by densitometry;

f) the protein fraction of the composition comprises greater than about 2% of a protein having a molecular weight of about 22,522 kDa and an isoelectric point of about 5.9 as assayed by two dimensional gel electrophoresis followed by quantitation by densitometry;

g) the protein fraction of the composition comprises greater than about 1% of a protein having a molecular weight of about 21,023 kDa and an isoelectric point of about 7.3 as assayed by two dimensional gel electrophoresis followed by quantitation by densitometry;

h) the protein fraction of the composition comprises greater than about 0.50% of a protein having a molecular weight of about 13,417 kDa and an isoelectric point of about 7.3 as assayed by two dimensional gel electrophoresis followed by quantitation by densitometry;

i) the protein fraction of the composition comprises a ratio of major protein constituents to minor protein constituents of less than 3.5:1 based on the aggregate mass of the proteins, wherein major protein constituents are the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry and the minor protein constituents are the remainder of the proteins;

j) the protein fraction of the composition comprises less than about 75% on a mass basis of the combined amounts of the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry;

k) the composition produces a solution having a color value of greater than 200 E 10%/1 cm when 250 mg of a dry powder of the composition are dissolved in one liter of water and absorbance is measured at 618 nm.

In some preferred embodiments, the PBP is Spirulina PC.

In some preferred embodiments, the composition has at least two of characteristics a, b, c, d, e, f, g, h, i, j and k. In some preferred embodiments, the composition has at least three of characteristics a, b, c, d, e, f, g, h, i, j and k. In some preferred embodiments, the composition has at least four of characteristics a, b, c, d, e, f, g, h, i, j and k. In some preferred embodiments, the composition has at least five of characteristics a, b, c, d, e, f, g, h, i, j and k. In some preferred embodiments, the composition has at least six of characteristics a, b, c, d, e, f, g, h, i, j and k. In some preferred embodiments, the composition has at least seven of characteristics a, b, c, d, e, f, g, h, i, j and k. In some preferred embodiments, the composition has at least eight of characteristics a, b, c, d, e, f, g, h, i, j and k. In some preferred embodiments, the composition has at least nine of characteristics a, b, c, d, e, f, g, h, i, j and k. In some preferred embodiments, the composition has at least ten of characteristics a, b, c, d, e, f, g, h, i, j and k. In some preferred embodiments, the composition has all eleven characteristics a, b, c, d, e, f, g, h, i, j and k.

In some preferred embodiments, the PBP composition is produced by a process comprising: encapsulating Spirulina to provide capsules; contacting the capsules with an aqueous medium under conditions such that the compound of interest passes from the capsule into the aqueous solution.

In some preferred embodiments, the PBP composition comprises a dried powder comprising a purified PBP, the powder having a residual moisture content of less than about 5% w/w of the powder.

In some preferred embodiments, the PBP composition is administered with a second biologically active agent. In some preferred embodiments, the second biologically active agent is a pharmaceutical agent. In some preferred embodiments, the second biologically active agent is an antiviral agent. In some preferred embodiments, the second biologically active agent is a nutraceutical agent.

In some preferred embodiments, the PBP composition is delivered as a topical formulation, an oral formulation, a mucosal formulation, an ophthalmological formulation, an aerosol formulation, and an intranasal formulation. In some preferred embodiments, the PBP composition is delivered via an oral delivery vehicle. In some preferred embodiments, the oral delivery vehicle is selected from the group consisting of a capsule, a tablet and a gummi gel. In some preferred embodiments, the PBP is delivered via a nebulizer or other positive pressure device. In some preferred embodiments, the PBP is delivered via a nasal spray device such as a metered spray device.

In some preferred embodiments, the PBP composition comprises a physiologically or pharmaceutically acceptable carrier.

In some preferred embodiments, the PBP composition used in a method or use as described above comprises an excipient selected from the group consisting of a buffer, a solubilizer, a preservative, an antioxidant, a humectant, a surfactant, a bioadhesive polymer, and a penetration enhancer and combinations thereof.

In some preferred embodiments, the present invention provides a nasal spray composition for administration of a PBP composition comprising an aqueous solution of a purified PBP composition and one or more excipients selected from the group consisting of a buffer, a solubilizer, a preservative, an antioxidant, a humectant, a surfactant, a bioadhesive polymer, and a penetration enhancer and combinations thereof. In some preferred embodiments, the present invention provides a metered dose spray device containing the nasal spray. In some preferred embodiments, the device comprises a nozzle insertable into the nasal cavity.

### DEFINITIONS

“Phycobiliprotein (PBP)” as used herein refers to photosynthetic antenna pigments in the cyanobacteria, red and cryptophyte algae, that efficiently harvest light energy, which is subsequently transferred to chlorophylls during photosynthesis. Phycobiliproteins are deeply colored, highly fluorescent, and water-soluble proteins with high propensity to form oligomers (hexamers) that constitute the building blocks of the extra-membranous antenna complex, phycobilisomes. The intensive color arises from covalently attached linear tetrapyrrole chromophores (phycobilins) via thioether bonds to the cysteine residues. Phycobilins are produced by heme metabolism. Heme is synthesized from protoheme IX by ferrochelatase. Then, heme oxygenase cleaves heme and biliverdin IXα is obtained. Biliverdin IXα is reduced by ferredoxin-dependent bilin reductases to obtain phycobilins. Final step in phycobiliproteins biosynthesis is the covalent attachment of bilin chromophores to the apoproteins, catalyzed by phycobiliprotein lyases. Slow spontaneous in vitro attachment of tetrapyrrole chromophores to the apoproteins has low fidelity and mixture of oxidation products is obtained.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Administering” or “administration of” a substance, a compound or an agent to a subject can be carried out using one of a variety of methods known to those skilled in the art. For example, a PBP can be administered, intravenously, arterially, intradermally, intra-muscularly, intraperitonealy, intravenously, subcutaneously, sublingually, orally (by ingestion), intranasally (by inhalation), intrapulmonary (by nebulization or instillation) intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

A “therapeutically effective amount” or a “therapeutically effective dose” of a drug or agent, such as PBP, is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of symptoms of the condition being treated, such as COVID-19. The skilled worker can readily determine the effective amount for a given situation by routine experimentation.

A “prophylactically effective amount” or a “prophylactically effective dose” of a drug or agent, such as PBP, is an amount of a drug or an agent that, when administered to a subject will have the intended prophylactic effect. The full prophylactic effect does not necessarily occur by administration of one dose, and may occur only after administration of a series of doses. Thus, a prophylactically effective amount may be administered in one or more administrations. The precise effective amount needed for a subject will depend upon, for example, the subject's size, health and age, the nature and extent of symptoms of the condition being treated, such as SARS-CoV-2 infection. The skilled worker can readily determine the effective amount for a given situation by routine experimentation.

“Treating” a condition or patient refers to taking steps to obtain beneficial or desired results, including clinical results. Beneficial or desired clinical results include, but are not limited to, alleviation, amelioration, or slowing the progression, of one or more symptoms associated with COVID-19. In certain embodiments, treatment may be prophylactic, such as for the prevention of infection by SARS-CoV-2.

### DETAILED DESCRIPTION OF THE INVENTION

Phycobiliproteins are water-soluble proteins that play a key role in photosynthesis. Phycobiliproteins take the form of a complex between proteins and covalently bound phycobilins that act as chromophores. They are the most important constituents of the phycobilisome. The major phycobiliproteins are C-phycocyanin, R-phycoerythrin, B-phycoerythrin and allophycocyanin. The blue phycobiliprotein (PBP) pigment complex contained in Spirulina that is generally called phycocyanin (PC) plays an important role in the photosynthetic chain acting as the link between light energy and chlorophyll. A number of different algae including Porphyridium cruentum, Galdieria sulphuria and Aphanizomenon flos aquae (AFA), other cyanobacteria and other organisms contain varying amounts of PBP complexes.

The present invention provides for the use of PBP compositions for the prevention or inhibition of infections by SARS-CoV-2 as well as the treatment of COVID-19 caused by SARS-CoV-2. In some preferred embodiments, the PBP is selected from C-phycocyanin, R-phycoerythrin, B-phycoerythrin and allophycocyanin. In some particularly preferred embodiments, the PBP is PC. In some preferred embodiments, treatment of COVID-19 comprises amelioration of one or more symptoms selected from dry cough, fever and shortness of breath. In some embodiments, compositions administered to a subject comprise a therapeutically effective amount of PBP, preferably Spirulina PBP. In some embodiments, a subject in need of treatment has been diagnosed as being infected by SARS-CoV-2 and/or as having COVID-19. In some embodiments, the PBP composition may be administered prophylactically to a subject in need thereof. For example, the PBP compositions may be administered to subjects that are at risk of exposure to SARS-CoV-2 infection such as health care workers, doctors, nurses, other care-givers, elderly subjects such as those over 60, 70 or 80 years of age, immunocompromised subjects, subjects with high blood pressure, and subjects with impaired lung function (e.g., subjects that have a history of lung cancer, chronic obstructive pulmonary disease (COPD), emphysema, smoking, or vaping).

In some embodiments, the subjects have long COVID symptoms. In some embodiments, the subject has exhibited long COVID symptoms for at least 2 months after diagnoses of COVID 19. In some embodiments, the subject has exhibited long COVID symptoms for at least 3 months after diagnoses of COVID 19. In some embodiments, the subject has exhibited long COVID symptoms for at least 4 months after diagnoses of COVID 19. In some embodiments, the subject has exhibited long COVID symptoms for at least 5 months after diagnoses of COVID 19. In some embodiments, the subject has exhibited long COVID symptoms for at least 6 months after diagnoses of COVID 19. In some embodiments, the subject has exhibited long COVID symptoms for at least 9 months after diagnoses of COVID 19. In some embodiments, the subject has exhibited long COVID symptoms for at least 12 months after diagnoses of COVID 19. In some embodiments, the long COVID symptoms are one or more of: fatigue, loss of smell and taste, shortness of breath, cough, joint pain, chest pain, difficulty with thinking and concentration (sometimes referred to as “brain fog”), depression, headache, heart palpitations, inflammation of the heart muscle, rash, hair loss, sleep disorders, loss of lung function and loss of memory. In some preferred embodiments, administration of the PBP composition improves or alleviates one or more of the following symptoms in a long COVID patient: fatigue, loss of smell and taste, shortness of breath, cough, joint pain, chest pain, difficulty with thinking and concentration (sometimes referred to as “brain fog”), depression, headache, heart palpitations, inflammation of the heart muscle, rash, hair loss, sleep disorders, loss of lung function and loss of memory.

In some embodiments, the PBP compositions are described in co-pending application Ser. No. 16/685,363, filed Nov. 15, 2019, and which is incorporated herein by reference in its entirety. In some preferred embodiments, the PBP is phycocyanin (PC).

The purification of PBP from organisms such as Spirulina is a delicate and potentially complex process, especially because these natural proteins are fragile and sensitive to heat and acids. For example, PBP is known to degrade rapidly at temperatures over 55° C. and at low PH, with a loss of blue color and consequently utility as a food colorant.

The destruction of the Spirulina cell wall, an integral part of the extraction of PBP, can damage the PBP and if the extraction takes place over a long period cause the degradation of the PBP. The nature of the raw biomass, for example whether it is fresh or dried, the type of drying process including temperature and time of exposure to heat, the preparation of the raw material, the addition of heat protecting substances, the length of time and temperature of storage, the final water content, the method of packaging, the level of oxidation of the protein and fats, exposure to bacteria and yeasts and fungi, residual chemicals and nutrients and range of other factors affect the quality and yield of PBP.

Once the PBP has been extracted, it has to be purified. Purification requires separation of blue- or other colored PBPs such as PC from the green-colored chlorophylls and is usually undertaken using filtration technologies including various types of centrifuges and filtration units. This can include disk centrifuges, decanting centrifuges, charcoal filtration, ultrafiltration, nanofiltration, reverse and forward osmosis filtration, high-pressure filtration, tangential filtration and a range of technologies that separate out the PC from the chlorophyll and cell debris and other undesirable substances.

Purification using filtration is an expensive process and contributes substantially to the high current cost of PBPs such as PC. Purification using filtration is also wasteful as it generally requires large quantities of disposable filters or filters that require cleaning with acid and alkaline solutions that are potential environmental pollutants if not managed and disposed of appropriately. Also, in order to reduce the cost of purification using filtration, additional pre-filtration production steps may be used to precipitate out undesirable substances using chitosan and other precipitating or flocculating or purifying agents. These steps are time-consuming, expensive and potentially polluting and can damage the PC. Also, the use of certain precipitating and flocculating and purifying agents may result in the PC no longer qualifying as being of organic origin, if the PC was purified from organic biomass.

Following purification, the liquid rich in PC is generally dried using spray drying processes. Prior to spray drying sugars such as trehalose or maltodextrin and salts such as sodium citrate may be added in order to facilitate the spray drying process, protect the PC from the heat of the spray drying process and enhance the color and solubility of the powder emanating from the spray drying process. As a result of the purification and drying steps, there is an inevitable loss in the yield and quality of the PC. This impacts on the quality, color value, pricing and profitability of the final product.

Other algae extracts are also extracted and in some cased purified and in some cases dried including AFA, a source of PC. For example, Chlorella extract or Chlorella Growth Factor (CGF) is generally extracted using high temperatures from Chlorella species and generally marketed without additional purification steps. Phycoerythrin, a red-colored PBP is extracted from Porphyridium cruentum, Gracilaria or red marine algae is an extremely valuable substance used in laboratories and cosmetics that is subjected to complex and expensive purification steps. Other algae such as Hematococcus pluvialis yield oil-soluble substances such as astaxanthin through extraction steps that include homogenization.

The form of delivery of the extracted and sometimes purified algae extract depends on its applications. For example PC as a food colorant is delivered as a dry powder. For human consumption as a nutraceutical, functional food, health food or pharmaceutical, there are processes to manufacture capsules containing Spirulina or PC preparations, to be swallowed. The document CN103285375 teaches the production of PC microspheres with an external oily layer, produced by the action of a calcium solution on an emulsion containing PC and sodium alginate in the water phase, paraffin and emulsifiers. The document CN101322568 describes capsules containing Spirulina, sodium alginate, chitosan, additives and calcium chloride. PC diffusion during storage is not mentioned and does not occur because the PC is degraded by heat during the pasteurization process.

In some preferred embodiments, PBP compositions useful in the present invention are produced by processes based on a chemical reaction between a gelling agent and a multivalent cation. Exposing the gelling agent to the multivalent cation (or visa versa) facilitates the formation of a gel and/or a membrane, trapping the cellular debris and other large-size molecular assemblies, but allowing diffusion of water-soluble small molecules and proteins into the surrounding aqueous medium, usually water, thereby permitting the purification of the desired substance such as the PBP or PC. The multivalent cation may also act on the cell wall of, for example Spirulina, thereby making it more fragile and porous and consequently allowing the extracellular diffusion of PBP and other water-soluble molecules. Thus, the methods of the present invention result not only in the purification of, for example PBP but also permit the extraction of PBP from Spirulina in an aqueous solution in a single step. The methods described may be utilized with other species of microalgae, including, but not limited to Chlorella, Porphyridium, Aphanizomenon flos aquae (AFA) and Galdieria.

In some preferred embodiments, the processes to prepare purified PBP from Spirulina comprises the following steps:
Mixing dried or fresh Spirulina, water and a gelling agent, for example sodium alginate to provide a first solution (Solution A)
Forming microcapsules by dropping small drops of Solution A using a dropper, pipette or some other industrial device or machine into a second solution of a salt of divalent cations.
Diluting the microcapsules into a predetermined volume of water or other liquid to provide an aqueous solution to permit purification through diffusion of the PBP from the microcapsules
Removal of the microcapsules and any debris from the liquid

In some preferred embodiments, the processes to prepare purified PBP from Spirulina comprises the following steps:
Mixing dried or fresh Spirulina, water and divalent cation, for example calcium chloride to provide a first solution (Solution A)
Forming microcapsules by dropping small drops of Solution A using a dropper, pipette or some other industrial device or machine into a second solution of a gelling agent, such as sodium alginate.
Diluting the microcapsules into a predetermined volume of water or other liquid to provide an aqueous solution to permit purification through diffusion of the PBP from the microcapsules
Removal of the microcapsules and any debris from the liquid

In some preferred embodiments, the processes to prepare purified PBP from Spirulina comprises the following steps:
Mixing dried or fresh Spirulina with water
Transforming the Spirulina and water mixture into a droplet, extruded tube or sausage or some other form
Coating this droplet, extruded tube or sausage or some other form with a layer of a solution of gelling agent such as sodium alginate
Coating the droplet, extruded tube or sausage or some other form that has been coated with the gelling agent, with a salt of divalent cations.
Placing the droplets, extruded tubes or sausages or some other forms that have been coated with the gelling agent and thereafter the divalent cation solution into a predetermined volume of water or other liquid to provide an aqueous solution to permit purification through diffusion of the PBP.
Removal of the droplets, extruded tubes or sausages or some other form and any debris from the liquid.

In some preferred embodiments, the different methods previously described to produce the purified PBP compositions can be used simultaneously, sequentially or repeatedly.

In some preferred embodiments, the PBP containing organism biomass is specially prepared through heat, cold, chemical and biological processes and physical mechanisms and techniques to facilitate the purification process of this invention.

In some preferred embodiments, the PBP composition comprises a dried powder comprising a purified PBP composition as described above, the powder having a residual moisture content of less than about 10% w/w of the powder.

In some preferred embodiments, the PBP composition comprises fresh or freshly harvested PBP containing organism biomass.

In some preferred embodiments, the microcapsule or other form contains chemicals or biological agents that enhance or retard or selectively control the purification of the PBP.

In some preferred embodiments, the microcapsule or other form contains a natural substance that is purified or concentrated in conjunction with or ‘chaperoned’ into the aqueous solution by the PBP or some other substance that exists in the organism biomass.

In some preferred embodiments the aqueous solution into which the PBP diffuses is modified by changing its temperature, through agitation or mixing or the addition of chemical or biological agents such as acids, alkalis, salts and antimicrobial agents to enhance or retard diffusion of PBP and to prevent contamination.

In some preferred embodiments, the microcapsule or other form is frozen, dried or treated in some other way to enhance or retard diffusion of the PBP into the aqueous solution

In some preferred embodiments, the compositions further comprise a second biologically active agent. In some preferred embodiments, the second biologically active agent is a pharmaceutical agent. In some preferred embodiments, the second biologically active agent is a nutraceutical agent. In some preferred embodiments, the second biologically active agent is an agricultural agent. In some preferred embodiments, the second biologically active agent is a cellular culture agent for plant, meat, poultry, porcine, bovine or other animal or plant or other organism cell culture.

In some preferred embodiments, the present invention provides an oral delivery vehicle comprising a PBP composition as described above. In some preferred embodiments, the oral delivery vehicle is selected from the group consisting of a capsule, a tablet and a gummi gel.

In some preferred embodiments, the present invention provides a delivery vehicle for agricultural applications and other applications such as bioremediation and soil amendment. In some preferred embodiments the delivery vehicle is a matrix or framework including natural and artificial matrices and frameworks including mycelia, fungal carpets, combinations of bacteria and other natural substances including ‘sympathetic combinations of bacteria and yeasts’ , matrices and frameworks based on nanotechnology, naturally existing plant matrices, collagen, bone and other tissue matrices and other natural or artificial materials to which the PBP composition can be attached, stuck, incorporated, bound or combined.

In some preferred embodiments, the microcapsule or other form contains, or is coated with, chemicals or biological agents that enhance or retard or selectively control the purification of the phycobiliprotein.

In some preferred embodiments, the microcapsule or other form contains a natural substance that is purified or concentrated in conjunction with or ‘chaperoned’ into the aqueous solution by the PBP or some other substance that exists in the organism biomass.

In some preferred embodiments, the microcapsule or other form is frozen, dried or treated in some other way to enhance or retard diffusion of the PBP into the aqueous solution

In some preferred embodiments, the biomass used is the genus Arthrospira, and more preferably to the species Arthrospira platensis (commonly known as Spirulina). In some preferred embodiments, the gelling agent is sodium alginate which reacts with most multivalent cations and especially well with the calcium ions. Preferred sources of calcium ions are, for example, calcium chloride and calcium gluconate. The present invention is not limited to the use of calcium ions. In other embodiments, the multivalent cations may be provided by salts of manganese, magnesium, zinc, or barium.

In some preferred embodiments, the first solution is prepared with 10% to 60% Spirulina (wet weight), 0.1% to 5% sodium alginate (dry weight) and water. Several additives can be added to the solution including flavorings, color agents, preservatives, moisteners, natural antibiotics, thickeners, sugars, anti-foaming agents, salts, acids and alkalis.

In some preferred embodiments, the second solution is an aqueous solution containing multivalent cations at a concentration between 0.005 and 0.5 mole per liter, preferably between 0.05 and 0.2 mole per liter (corresponding for example to the range 5.5-22 g/L of calcium chloride). This solution can also contain flavorings, color additives, preservatives and acidifying agents.

The present invention is not limited to any particular mechanism of action. Indeed, an understanding of the mechanism of action is not necessary to practice the invention. Nevertheless, it is contemplated that the microcapsules generated by dropping the first solution into the second solution are irregular, roughly egg-shaped with a maximum dimension of less than 6 mm and have a solid texture. Other forms can include tubular or spaghetti-like shapes, sausages, disks and irregular shapes. The setting time, during which the microcapsules or other forms are immersed in the solution B, is preferably less than 1 hour.

The purification of the preferably takes place as the last step of the process. Specifically, the microcapsules are immersed in a large volume of water and the medium containing the microcapsules is kept at a low temperature (between 0 and 6° C.) for from about 1 to 60 hours. The diffusion of the purified PBP can demonstrated when the water become progressively bluer and shows a purple fluorescence when exposed to light. These observed phenomena as characteristic of the PBP complex of Spirulina.

In preferred embodiments, the microcapsules or other forms may be manufactured in a workshop or a factory and the extraction is undertaken at a consumer's home or at an industrial site or on a farm or in a laboratory.

In some preferred embodiments, microcapsules are packaged into a porous container such as a bag, a sachet, a pod or the like, with a pore size much lower than the microcapsule size to ensure that PBP can diffuse while the microcapsules remain in the container. In some preferred embodiments, the process steps are conducted at low temperatures (e.g., between 0 and 6° C.) to prevent PBP degradation or natural or chemical preservatives are added.

The process of purification described in this invention has a number of advantages over existing algal biomass purification methods. The main advantage is that it is possible to limit or even avoid the use of membrane-based filtration in the production of purified algal extracts such as PC, thereby substantially reducing the cost of production and improving the quality of such extracts. The process described also does not require the use of salts and other substances to precipitate out the PC and the consequent expense of dialysis, osmosis, gels and exchange columns to remove such salts. This invention also can increase the yield of PBP extraction and the concentration of proteins of interest in the purified extract. This invention permits the combination of extraction and purification in a single step, if desired. As a result, it is possible to make purified extract of Spirulina with very high PC concentrations. This invention permits purification of PBP without using chemical solvents or any product from animal origin, allowing the making of an PC-rich extract compatible with vegan, halal and kosher food requirements. This invention is not damaging to delicate and sensitive proteins and other molecules including PBP thereby permitting the purification of highly active natural extracts. This invention permits low-waste, low water consumption purification with positive impacts in terms of sustainability and compatibility with circular economy principles. The processes can be applied to a wide range of algae and other natural substances. This invention is easily scalable with fairly low capital expenditure costs on machinery and technologies.

The methods described herein are useful for producing compositions containing a high quality protein fraction that is enriched for PBP. The protein content and quality of the PBP purified extracts obtained by these methods differ substantially from other described PBP purified extracts, generally purified using membrane filtration methods.

Accordingly, in some preferred embodiments, the present invention provides purified PBP protein compositions characterized by one or more of the following characteristics:

a) the protein fraction of the composition comprises greater than about 30% of a protein having a molecular weight of about 17,695 kDa and an isoelectric point of about 6.29 as assayed by two dimensional gel electrophoresis followed by quantitation by densitometry;

b) the protein fraction of the composition comprises greater than about 5% of a protein having a molecular weight of about 19,833 kDa and an isoelectric point of about 6.14 as assayed by two dimensional gel electrophoresis followed by quantitation by densitometry;

c) the protein fraction of the composition comprises greater than about 1% of a protein having a molecular weight of about 45,578 kDa and an isoelectric point of about 6.2 as assayed by two dimensional gel electrophoresis followed by quantitation by densitometry;

d) the protein fraction of the composition comprises greater than about 1% of a protein having a molecular weight of about 35,014 kDa and an isoelectric point of about 5.9 as assayed by two dimensional gel electrophoresis followed by quantitation by densitometry;

e) the protein fraction of the composition comprises greater than about 0.30% of a protein having a molecular weight of about 24,688 kDa and an isoelectric point of about 5.3 as assayed by two dimensional gel electrophoresis followed by quantitation by densitometry;

f) the protein fraction of the composition comprises greater than about 2% of a protein having a molecular weight of about 22,522 kDa and an isoelectric point of about 5.9 as assayed by two dimensional gel electrophoresis followed by quantitation by densitometry;

g) the protein fraction of the composition comprises greater than about 1% of a protein having a molecular weight of about 21,023 kDa and an isoelectric point of about 7.3 as assayed by two dimensional gel electrophoresis followed by quantitation by densitometry;

h) the protein fraction of the composition comprises greater than about 0.50% of a protein having a molecular weight of about 13,417 kDa and an isoelectric point of about 7.3 as assayed by two dimensional gel electrophoresis followed by quantitation by densitometry;

i) the protein fraction of the composition comprises a ratio of major protein constituents to minor protein constituents of less than 3.5:1 based on the aggregate mass of the proteins, wherein major protein constituents are the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry and the minor protein constituents are the remainder of the proteins;

j) the protein fraction of the composition comprises less than about 75% on a mass basis of the combined amounts of the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry; and

k) the composition produces a solution having a color value of greater than 180 E 10%/1cm when 250 mg of a dry powder of the composition are dissolved in one liter of water and absorbance is measured at 618 nm.

In some preferred embodiments, the composition has at least two of characteristics a, b, c, d, e, f, g, h, i, j and k. In some preferred embodiments, the composition has at least three of characteristics a, b, c, d, e, f, g, h, i, j and k. In some preferred embodiments, the composition has at least four of characteristics a, b, c, d, e, f, g, h, i, j and k. In some preferred embodiments, the composition has at least five of characteristics a, b, c, d, e, f, g, h, i, j and k. In some preferred embodiments, the composition has at least six of characteristics a, b, c, d, e, f, g, h, i, j and k. In some preferred embodiments, the composition has at least seven of characteristics a, b, c, d, e, f, g, h, i, j and k. In some preferred embodiments, the composition has at least eight of characteristics a, b, c, d, e, f, g, h, i, j and k. In some preferred embodiments, the composition has at least nine of characteristics a, b, c, d, e, f, g, h, i, j and k. In some preferred embodiments, the composition has at least ten of characteristics a, b, c, d, e, f, g, h, i, j and k. In some preferred embodiments, the composition has all eleven characteristics a, b, c, d, e, f, g, h, i, j and k. It will be understood by those of skill in the art that in preferred embodiments, the compositions of the present invention may be identified by any subcombination of one or more of the characteristics identified above.

In some preferred embodiments, the purified phycobiliprotein composition is produced by a process comprising: mixing PBP containing organism biomass with water and gelling agent, forming a droplet, introducing a droplet of the first solution into a second solution containing a salt of divalent cations under conditions such that microcapsules form, and obtaining an extract enriched for PBP by mixing the microcapsules with a volume of an aqueous solution under conditions such that the phycobiliprotein diffuses from the microcapsules into the aqueous solution.

In some preferred embodiments, the purified PBP composition is produced by a process comprising: mixing PBP containing organism biomass with water and a salt of divalent cations, forming a droplet, introducing a droplet of the first solution into a second solution containing a gelling agent under conditions that microcapsules form, and obtaining an extract enriched for PBP by mixing the microcapsules with a volume of an aqueous solution under conditions such that the PBP diffuses from the microcapsules into the aqueous solution.

In some preferred embodiments, the purified PBP composition is produced by a process comprising: mixing PBP containing organism biomass with water, coating a droplet, extruded tube, sausage or some other form of this first solution with a second solution of gelling agent and introducing a droplet, extruded tube, sausage or some other form of the first solution coated with the second solution into a third solution containing a salt of divalent cations under conditions such that a coated droplet, extruded tube sausage or another form is obtained, and thereafter obtaining purified extract rich in PBP by mixing the droplet, extruded tube, sausage or other form with a volume of an aqueous solution under conditions such that the PBP diffuses from the droplet, extruded tube, sausage or other form into the aqueous solution.

In some preferred embodiments, the different methods previously described to produce the purified PBP compositions can be used simultaneously, sequentially or repeatedly.

In some preferred embodiments, the PBP compositions are provided as a dried powder. In some preferred embodiments, the residual moisture in the powder is less than 5%, more preferably less than 4%, and most preferably less than 3% or 1%. In other preferred embodiments, the powder may be produced by spray-drying, spray-freeze drying, refractance window drying, microwave drying, air drying, fluidized bed drying, vacuum drying, natural drying, microwave drying or foam drying the solutions of purified PBP.

Pharmaceutical and nutraceutical compositions of the present invention preferably comprise an effective amount of a PBP composition, such as the dried powder.

The phrases “pharmaceutical or physiologically acceptable” refers to molecular entities and compositions that do not produce an adverse, allergic or other untoward reaction when administered to an animal, such as, for example, a human, as appropriate. The preparation of a pharmaceutical composition that contains at least one compound or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically of physiologically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The PBP compositions of the present invention may be formulated with different types of carriers depending on whether it is to be administered in solid, liquid or aerosol form, and whether it need to be sterile for such routes of administration as injection. The PBP compositions of the present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation and nebulization (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

Upon formulation, PBP compositions of the present invention will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the PBP composition of the present invention suitable for administration is provided in a physiologically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the PBP composition is combined with the carrier in any convenient and practical manner, i.e., by solution, suspension, emulsification, admixture, encapsulation, absorption and the like. Such procedures are routine for those skilled in the art.

Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration: Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical or nutraceutical compositions may comprise, for example, at least about 0.1% of the PBP composition, such as a powdered PBP composition. In other embodiments, the PBP composition, such as a powdered PBP composition may comprise between about 2% to about 75% of the weight of the unit, or between about 25% to about 60%, for example, and any range derivable therein. Naturally, the amount of the PBP composition, such as a powdered PBP composition in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose of the PBP composition, such as a PBP powder composition, may also comprise from about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In preferred embodiments of the present invention, the PBP composition is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the PBP compositions may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as; for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the PBP compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In a specific embodiment of the present invention, the composition is combined or mixed thoroughly with a semi-solid or solid carrier. In some preferred embodiments, the lipid compositions are incorporated into chewable matrices. Preferred chewable matrices jelly candies and gelatin-based gummi candy. Exemplary gummi candies include gummi bears, gummi worms, gummi frogs, gummi hamburgers, gummi cherries, gummi soda bottles, gummi sharks, gummi army men, gummi hippopotami, gummi lobsters, gummi watermelons, gummi octopuses, gummi apples, gummi peaches, and gummi oranges. The terms “gummi” and “gummy” are used interchangeably herein.

In some particularly preferred embodiments, the chewable matrix material is a sweetened material commonly referred to a gummy candy or jelly material. Gummy candy or jelly sweets are a broad general type of gelatin based, chewy candy. Gummy bears are the most popular and well known of the gummy candies. Other shapes are provided as well and gummy candies are sometimes combined with other forms of candy such as marshmallows and chocolates and as well made sour.

In preferred embodiments, the chewable matrix material comprises a gelling agent, which may be any physiologically tolerable gelling agent (preferably a saccharide (e.g. an oligosaccharide or polysaccharide), a protein or a glycoprotein) or combination capable of forming a soft, chewable, self-supporting chewable gel. Many such materials are known from the food and pharmaceutical industry and are discussed for example in Handbook of hydrocolloids, G O Phillips and P A Williams (Eds.), Woodhead Publishing, Cambridge, UK, 2000. The gelling agents are preferably materials capable of undergoing a sol-gel transformation, e.g. under the influence of a change in physiochemical parameters such as temperature, pH, presence of metal ions (e.g. group 1 or 2 metal ions), etc. Preferred gelling agents include gelatins, alginates and carrageenans. However, the use of gelatins is especially preferred as breakdown in the throat of trapped fragments is ensured and as cores having the desired properties may readily be produced using gelatins.

The gelatins used as gelling agents in the chewable matrix of the invention may be produced from the collagen of any mammal or the collagen of any aquatic species, however the use of gelatin from salt-water fish and in particular cold and warm water fishes is preferred. Gelatins having an amino acid content of 5 to 25% wt. are preferred, more especially those having an amino acid content of 10 to 25% wt. The gelatins will typically have a weight average molecular weight in the range 10 to 250 kDa, preferably 75 to 220 kDa, especially 80 to 200 kDa. Gelatins having no Bloom value or low Bloom values of 60-300, 150-300 and especially 90-200 are preferred. Where a gelatin of no Bloom value, e.g. a cold water fish gelatin, is used, this will typically be used together with another gelatin or other gelling agent. The combination of cold water and warm water fish gelatins is especially preferred. The gelatin will typically be present in the aqueous phase at a concentration of 1 to 50% wt., preferably 2 to 35% wt., particularly 5 to 25% wt. In the case of mixtures of gelatin and polysaccharides, the weight ratio of gelatin to polysaccharide in the aqueous phase will typically be 50:1 to 5:1, preferably 40:1 to 9:1, especially 20:1 to 10:1.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides, or combinations thereof. In certain embodiments, suppositories may be formed from mixtures containing, for example, the active ingredient in the range of about 0.5% to about 10%, and preferably about 1% to about 2%.

In further embodiments, the PBP compositions may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the PBP compositions may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the PBP compositions in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In other preferred embodiments of the invention, the active compound may be formulated for administration via other routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Preferred bases for compositions for topical application include polyethylene glycol, lanolin, cold cream and petrolatum as well as any other suitable absorption, emulsion or water-soluble ointment base. Preferred topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain preferred embodiments, the pharmaceutical compositions may be delivered by eye drops, intranasal sprays, inhalation, via nebulization in continuous positive pressure and other forms of ventilation using a variety of devices, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

In some preferred embodiments, the PBP compositions are provided in a nasal spray. In some preferred embodiments, the nasal sprays comprise one or more excipients selected from a buffer, a solubilizer, a preservative, an antioxidant, a humectant, a surfactant, a bioadhesive polymer, and a penetration enhancer. Examples of buffers useful in nasal sprays of the invention include sodium phosphate, sodium citrate, and citric acid. Examples of solubilizers useful in nasal sprays of the invention include solvents or co-solvents such as glycols, alcohol, Transcutol (diethylene glycol monoethyl ether), medium chain glycerides, Labrasol (saturated polyglycolyzed C8-C10 glyceride) and cyclodextrin. Examples of preservatives useful in nasal sprays of the invention include parabens, phenyl ethyl alcohol, benzalkonium chloride, EDTA and benzoyl alcohol. Examples of antioxidants useful in nasal sprays of the invention include sodium bisulfite, butylated hydroxytoluene, sodium metabisulfite and tocopherol. Examples of humectants useful in nasal sprays of the invention include glycerin, sorbitol and mannitol. Examples of surfactants useful in nasal sprays of the invention include polysorbet as well as surfactants described elsewhere herein.

In some preferred embodiments, the pH of a nasal spray comprising PBP of the present invention is from pH 5.0 to 6.5. In some preferred embodiments, the osmolality of a nasal spray comprising PBP of the present invention is from 100 or 600 mOsmol/Kg

In some embodiments, the nasal spray comprising a PBP composition is provided in a metered-dose spray pump. Metered spray pumps of the present invention pumps preferably deliver 100 μl (25-200 μl) per spray. In some preferred embodiments, the device comprises a nozzle insertable into the nasal cavity.

In some embodiments, the PBP compositions are formulated for oral administration with flavoring agents or sweeteners. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate. Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product. In addition to the carbohydrates described above, the nutritional supplement can contain natural or artificial (preferably low calorie) sweeteners, e.g., saccharides, cyclamates, aspartamine, aspartame, acesulfame K, and/or sorbitol.

The PBP compositions of the present invention may also be delivered as nutraceuticals, dietary supplements, nutritional supplements, or functional foods.

The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like. For example, the dietary supplement of the present invention may contain one or more of the following: ascorbates (ascorbic acid, mineral ascorbate salts, rose hips, acerola, and the like), dehydroepiandosterone (DHEA), green tea (polyphenols), inositol, kelp, dulse, bioflavonoids, maltodextrin, nettles, niacin, niacinamide, rosemary, selenium, silica (silicon dioxide, silica gel, horsetail, shavegrass, and the like), spirulina, zinc, and the like. Such optional ingredients may be either naturally occurring or concentrated forms.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

In other embodiments, the present invention provides nutritional supplements (e.g., energy bars or meal replacement bars or beverages) comprising of the PBP compositions of the present invention. In preferred embodiments, the nutritional supplements comprise an effective amount of the components as described above. The nutritional supplement may serve as meal or snack replacement and generally provide nutrient calories. Preferably, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, wherein simple carbohydrates are mono- or disaccharides. The nutritional supplement contains, in one embodiment, combinations of sources of carbohydrate of three levels of chain length (simple, medium and complex; e.g., sucrose, maltodextrins, and uncooked cornstarch).

In still further embodiments, the present invention provides food products, prepared food products, or foodstuffs (i.e., functional foods) comprising the PBP compositions of the present invention. In preferred embodiments, the foods comprise an effective amount of the components as described above. For example, in some embodiments, beverages and solid or semi-solid foods comprising the fatty acids or derivatives thereof are provided. These forms can include, but are not limited to, beverages (e.g., soft drinks, milk and other dairy drinks, and diet drinks), baked goods, puddings, dairy products, confections, snack foods, or frozen confections or novelties (e.g., ice cream, milk shakes), prepared frozen meals, candy, snack products (e.g., chips), soups, spreads, sauces, salad dressings, prepared meat products, cheese, yogurt and any other fat or oil containing foods, and food ingredients (e.g., wheat flour).

In some preferred embodiments, the PBP compositions of the present invention are provided in a formulation with a stabilizing agent and/or antioxidant that does not naturally occur in Spirulina. Preferred stabilizing agents are described above. As used herein, the term “antioxidant” is recognized in the art and refers to synthetic or natural substances that prevent or delay the oxidative deterioration of a compound. In preferred embodiments, antioxidants that may be used in the PBP compositions of the present invention include tocopherols, flavonoids, catechins, superoxide dismutase, lecithin, gamma oryzanol; vitamins, such as vitamins A, C (ascorbic acid) and E and beta-carotene; natural components such as camosol, carnosic acid and rosmanol found in rosemary and hawthorn extract, proanthocyanidins such as those found in grapeseed or pine bark extract, and green tea extract.

In some preferred embodiments, the PBP compositions of the present invention are co-formulated or co-administered with one or more additional bioactive agents. In preferred embodiments, the one or more additional bioactive agents are selected from nutraceutical and pharmaceutical agents.

In preferred embodiments, the nutraceutical agents are selected from biologically active lipids (e.g., eicosapentaenoic acid (EPA), docosahexaenoic acid (DHA), conjugated linoleic acid, omega-3 fatty acids, polyunsaturated fatty acids, short chain fatty acids, medium chain fatty acids, in free fatty acid, triglyceride or phospholipid forms), resveratrol, coenzyme q10 (ubiquinone), melatonin, chondroitin sulfate, glucosamine, s-adenosylmethionine, astaxanthin, carnitine, tryptophan, alpha-lipoic acid, glutamine, inositol, green tea extract, flavonoids (e.g., epi-gallo catechin gallate (EGCG), epi-gallo catechin (EGC) and epi-catechin (EC), green tea extract, luteins), terpenes, gallates, quercertin, aequorin, and curcumin.

In preferred embodiments, pharmaceutical agents are selected from the group consisting of antiviral and anti-inflammatory drugs.

In some preferred embodiments, exemplary antiviral drugs suitable for use in formulations of the present invention include, but are not limited to Chloroquine, Hydroxychloroquine, Remdesivir, Favipiravir, Abacavir, Aciclovir, Acyclovir, Adefovir, Amantadine, Amprenavir, Ampligen, Arbidol, Atazanavir, Atripla, Boceprevir, Cidofovir, Combivir, Darunavir, Delavirdine, Didanosine, Docosanol, Edoxudine, Efavirenz, Emtricitabine, Enfuvirtide, Entecavir, Famciclovir, Fomivirsen, Fosamprenavir, Foscarnet, Fosfonet, Ganciclovir, Ibacitabine, Imunovir, Idoxuridine, Imiquimod, Indinavir, Inosine, Lamivudine, Lopinavir, Loviride, Maraviroc, Moroxydine, Methisazone, Nelfinavir, Nevirapine, Nexavir, Oseltamivir (Tamiflu), Peginterferon alfa-2a, Penciclovir, Peramivir, Pleconaril, Podophyllotoxin, Raltegravir, Ribavirin, Rimantadine, Ritonavir, Pyramidine, Saquinavir, Stavudine, Tea tree oil, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza) and Zidovudine.

In some embodiments, exemplary non-steroidal anti-inflammatory drugs (NSAIDs) suitable for use in formulations of the present invention include, but are not limited to Choline salicylate (Arthropan) Celecoxib (Celebrex); Diclofenac potassium (Cataflam); Diclofenac sodium (Voltaren, Voltaren XR); Diclofenac sodium with misoprostol (Arthrotec); Diflunisal (Dolobid); Etodolac (Lodine, Lodine XL); Fenoprofen calcium (Nalfon); Flurbiprofen (Ansaid); Ibuprofen (Advil, Motrin, Motrin IB, Nuprin); Indomethacin (Indocin, Indocin SR); Ketoprofen (Actron, Orudis, Orudis KT, Oruvail); Magnesium salicylate (Arthritab, Bayer Select, Doan's Pills, Magan, Mobidin, Mobogesic); Meclofenamate sodium (Meclomen); Mefenamic acid (Ponstel); Meloxicam (Mobic); Nabumetone (Relafen); Naproxen (Naprosyn, Naprelan); Naproxen sodium (Aleve, Anaprox); Oxaprozin (Daypro); Piroxicam (Feldene); Rofecoxib (Vioxx); Salsalate (Amigesic, Anaflex 750, Disalcid, Marthritic, Mono-Gesic, Salflex, Salsitab); Sodium salicylate (various generics); Sulindac (Clinoril); Tolmetin sodium (Tolectin); and Valdecoxib (Bextra).

In some embodiments the PBP compositions can be presented for applications in the food and beverage industry as a powder or solution with or without salts, sugars, natural preservatives, chemicals or other substances.

In some embodiments the PBP compositions can be presented in high-sugar solutions used for panning or coating hard and soft candies, enrobing chocolates, preparation of sweets, candies and desserts and other applications in the confectionary, baking, food and dairy industries

In some embodiments the PBP compositions can be presented for applications in agriculture and animal nutrition in powder and liquid form with or without the addition of chemicals, vitamins, preservatives, sugars, medications, drugs, hormones, biological agents, surfactants, spreading and dissolution agents for uses that include all forms of irrigation, foliar spraying, hydroponics, animal feed and nutritional supplementation, fish feed and nutritional supplementation, fresh and sea water organism feed and nutritional supplementation, mushroom farming, mycelial growing, crustacean feed and nutritional supplementation, domestic animal food and treats and nutraceuticals and functional foods and medications, organic farming applications, biodynamic farming applications and regenerative agriculture applications.

In some embodiments the PBP compositions can be presented in liquid and powder form with or without: other algal compositions some purified according to this invention, chemicals, vitamins, preservatives, sugars, medications, drugs, hormones, biological agents, surfactants, spreading and dissolution agents for applications in bioremediation, pollution control, soil depollution, water treatment and water depollution.

In some embodiments the PBP compositions can be presented in liquid and powder form with or without: other algal compositions some purified according to this invention chemicals, vitamins, preservatives, sugars, medications, drugs, hormones, biological agents, surfactants, spreading and dissolution agents for applications in cell culture, cellular agriculture, micro-propagation and other techniques for growing cells of all living organisms including bacteria, fungi, plants, rodents, mammals, bird, fish, crustacean and other marine and terrestrial organisms.

1. A method of treating COVID-19 or infection by SARS-CoV-2 in a subject in need thereof, comprising administering to a subject diagnosed with COVID-19 a therapeutically effective amount of a phycobiliprotein (PBP) composition.

2. A method of inhibiting SARS-CoV-2 infection in a subject in need thereof comprising administering to a subject diagnosed with COVID-19 a prophylactically effective amount of a phycobiliprotein (PBP).

3. The method of claim 2, wherein the subject in need thereof is selected from the group consisting of a health care workers, a doctor, a nurse, a teacher, a day-care worker, a hospital worker, elderly subjects such as those over 60, 70 or 80 years of age, an immunocompromised subject, a subject with high blood pressure, and a subject with impaired lung function.

4. The method of claim 3, wherein the subject with impaired lung function has a history of lung cancer, chronic obstructive pulmonary disease (COPD), emphysema, smoking, or vaping.

5-8. (canceled).

9. Method of claim 1, wherein the subject has long COVID-19.

10. Method of claim 9, wherein the subject has exhibited long COVID symptoms for at least 3 months, 4 months, 5 months, six months, 9 months or 12 months after diagnoses of COVID 19. In some embodiments, the subject has exhibited long COVID symptoms for at least 4 months after diagnoses of COVID 19.

11. Method of claim 10, wherein the long COVID symptoms are one or more of: fatigue, loss of smell and taste, shortness of breath, cough, joint pain, chest pain, difficulty with thinking and concentration, depression, headache, heart palpitations, inflammation of the heart muscle, rash, hair loss, sleep disorders, loss of lung function and loss of memory.

12. Method or claim 1, wherein the PBP composition is a purified phycobiliprotein (PBP) composition characterized by one or more of the following characteristics:
a) the protein fraction of the composition comprises greater than about 30% of a protein having a molecular weight of about 17,695 kDa and an isoelectric point of about 6.29 as assayed by two dimensional gel electrophoresis followed by quantitation by densitometry;
b) the protein fraction of the composition comprises greater than about 5% of a protein having a molecular weight of about 19,833 kDa and an isoelectric point of about 6.14 as assayed by two dimensional gel electrophoresis followed by quantitation by densitometry;
c) the protein fraction of the composition comprises greater than about 1% of a protein having a molecular weight of about 45,578 kDa and an isoelectric point of about 6.2 as assayed by two dimensional gel electrophoresis followed by quantitation by densitometry;
d) the protein fraction of the composition comprises greater than about 1% of a protein having a molecular weight of about 35,014 kDa and an isoelectric point of about 5.9 as assayed by two dimensional gel electrophoresis followed by quantitation by densitometry;
e) the protein fraction of the composition comprises greater than about 0.30% of a protein having a molecular weight of about 24,688 kDa and an isoelectric point of about 5.3 as assayed by two dimensional gel electrophoresis followed by quantitation by densitometry;
f) the protein fraction of the composition comprises greater than about 2% of a protein having a molecular weight of about 22,522 kDa and an isoelectric point of about 5.9 as assayed by two dimensional gel electrophoresis followed by quantitation by densitometry;
g) the protein fraction of the composition comprises greater than about 1% of a protein having a molecular weight of about 21,023 kDa and an isoelectric point of about 7.3 as assayed by two dimensional gel electrophoresis followed by quantitation by densitometry;
h) the protein fraction of the composition comprises greater than about 0.50% of a protein having a molecular weight of about 13,417 kDa and an isoelectric point of about 7.3 as assayed by two dimensional gel electrophoresis followed by quantitation by densitometry;
i) the protein fraction of the composition comprises a ratio of major protein constituents to minor protein constituents of less than 3.5:1 based on the aggregate mass of the proteins, wherein major protein constituents are the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry and the minor protein constituents are the remainder of the proteins;
j) the protein fraction of the composition comprises less than about 75% on a mass basis of the combined amounts of the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry;
k) the composition produces a solution having a color value of greater than 200 E 10%/1 cm when 250 mg of a dry powder of the composition are dissolved in one liter of water and absorbance is measured at 618 nm.

13-22. (canceled).

23. Method of claim 1, wherein the PBP composition is produced by a process comprising:
encapsulating Spirulina to provide capsules;
contacting the capsules with an aqueous medium under conditions such that the compound of interest passes from the capsule into the aqueous solution.

24. Method of claim 1 wherein the PBP composition comprises a dried powder comprising a purified PBP, the powder having a residual moisture content of less than about 5% w/w of the powder.

25-28. (canceled).

29. Method of claim 1, wherein the PBP composition is delivered as a topical formulation, an oral formulation, a mucosal formulation, an ophthalmological formulation, an aerosol formulation, and an intranasal formulation.

30-31. (canceled).

32. Method of claim 1, wherein the PBP is delivered via a nebulizer or other positive pressure device.

33. Method of claim 1, wherein the PBP is delivered via a nasal spray device.

34. Method of claim 1, wherein the PBP composition comprises a physiologically or pharmaceutically acceptable carrier.

35. Method of claim 1, wherein the PBP composition comprises an excipient selected from the group consisting of a buffer, a solubilizer, a preservative, an antioxidant, a humectant, a surfactant, a bioadhesive polymer, and a penetration enhancer and combinations thereof.

36. A nasal spray composition for administration of a PBP composition comprising an aqueous solution of a purified PBP composition and one or more excipients selected from the group consisting of a buffer, a solubilizer, a preservative, an antioxidant, a humectant, a surfactant, a bioadhesive polymer, and a penetration enhancer and combinations thereof.

37-38. (canceled).
